(19) United States
(12) Patent Application Publication
(10) Pub. No.: US 2022/0080075 A1
(43) Pub. Date: Mar. 17, 2022

(54) System and Method for Verifying Cleaning

(71) Applicant: Spotcheck, Inc., Bensenville, IL (US)

(72) Inventors: Brian A. Larson, Chicago, IL (US); Jeffrey M. Banasek, Lombard, IL (US)

(21) Appl. No.: 17/473,088

(22) Filed: Sep. 13, 2021

Related U.S. Application Data
(60) Provisional application No. 63/077,146, filed on Sep. 11, 2020.

(51) Int. Cl. A61L 2/28; G01N 21/94

(52) U.S. Cl. CPC A61L 2/28; A61L 2202/14; G01N 21/94

(57) ABSTRACT

A method and kit are provided for training a cleaning protocol and verifying the efficacy of cleaning performed on a surface. The method may include applying a marking agent to a surface to be cleaned. The surface may be tested for the presence of the marking agent after an expected cleaning of the surface. The presence of the marking agent on the surface after the expected cleaning of the surface may be indicative of a lack of cleaning of the surface or an ineffective cleaning of the surface.

### CROSS-REFERENCE TO RELATED APPLICATION

This application claims the benefit of U.S. provisional application Ser. No. 63/077,146, filed on Sep. 11, 2020, entitled “System and Method for Verifying Cleaning,” the entire disclosure of which is incorporated herein by reference.

### TECHNICAL FIELD

The present disclosure generally relates to surface cleaning, and more particularly relates to systems and methods for determining whether a surface has been cleaned and how thoroughly a surface has been cleaned.

### BACKGROUND

A proper cleaning protocol can provide a powerful first line of defense against infectious disease transmission, particularly viral disease transmission, as well as mitigating the risk of spreading potentially hazardous contaminants, such as hazardous drugs or other contaminants, from one surface to another or from a surface to individuals coming in contact with contaminated surfaces.

### SUMMARY

According to an implementation, a method may include applying a marking agent to a surface to be cleaned. The method may also include testing the surface for the presence of the marking agent on the surface after an expected cleaning of the surface.

One or more of the following features may be included. Applying the marking agent to the surface may include spraying the marking agent on to the surface. Applying the marking agent to the surface may include wiping the surface with an applicator including the marking agent. Applying the marking agent to the surface may include dusting the surface with the marking agent. Wiping the surface with an applicator may include dispensing the marking agent onto the applicator and wiping the surface with the applicator. Wiping the surface with an applicator may include wiping the surface with a pretreated applicator including the marking agent.

The marking agent may not be readily visually perceptible after application to the surface. The marking agent may include an acid. The marking agent may include a mixture of the acid and a carrier fluid.

Testing may include testing after an anticipated cleaning of the surface. Testing the surface for the presence of the marking agent may include contacting the surface with an indicator agent.

Contacting the surface with an indicator agent may include spraying the surface with the indicator agent. Contacting the surface with an indicator agent may include wiping the surface with an applicator including the indicator agent. Wiping the surface with an applicator may include dispensing the indicator agent onto the applicator and wiping the surface with the applicator. Wiping the surface with an applicator may include wiping the surface with a pretreated applicator including the indicator agent.

The indicator agent may provide a visually perceptible indication of the presences of the marking agent on the surface. The marking agent may include an acid, and the indicator agent may include an acid/base indicator. Detecting the presence of the marking agent on the surface after an expected cleaning of the surface may indicate at least one of a lack of the expected cleaning and an ineffectiveness of the expected cleaning.

According to another implementation, a cleaning protocol verification kit may include a marking agent dispenser configured for applying a marking agent to a surface. The cleaning protocol verification kit may also include an indicator agent dispenser configured for contacting an indicator agent with the surface. The indicator agent may provide a visually perceptible indication of the presences of the marking agent on the surface for one or more of training users to implement a cleaning protocol and verifying an efficacy of cleaning of the surface.

One or more of the following features may be included. The marking agent dispenser may include a spray bottle containing the marking agent, and may be configured to dispense a metered amount of marking agent onto the surface. The marking agent dispenser may include a spray bottle containing the marking agent, and may be configured to dispense a metered amount of marking agent onto an applicator. The marking agent dispenser may include an applicator pretreated with the marking agent.

The indicator agent dispenser may include a spray bottle containing the indicator agent, and may be configured to dispense a metered amount of the indicator agent onto the surface. The indicator agent dispenser may include a spray bottle containing the indicator agent, and may be configured to dispense a metered amount of the indicator agent onto an applicator. The indicator agent dispenser may include an applicator pretreated with the indicator agent.

The marking agent may include an acid, and the indicator agent may include an acid/base indicator. The acid/base indicator may be configured to provide a visually perceptible change when contacted with the marking agent.

### BRIEF DESCRIPTION OF THE DRAWINGS

FIG. 1 diagrammatically depicts a surface having an indication of a marking agent, according to an illustrative example embodiment.

FIG. 2 diagrammatically depicts an applicator indicating the presence of a marking agent on a region contacted by the applicator, according to an illustrative example embodiment.

FIG. 3A diagrammatically depicts a spray bottle dispenser for a marking agent and/or an indicator agent, according to an illustrative example embodiment.

FIG. 3B diagrammatically depicts an applicator for a marking agent and/or an indicator agent, such as a cloth, wipe, paper towel, towelette, etc., according to an illustrative example embodiment.

FIG. 3C diagrammatically depicts a swab applicator for a marking agent and/or an indicator agent, according to an illustrative example embodiment.

### DESCRIPTION OF EXAMPLE EMBODIMENTS

In general, the present disclosure relates to verifying cleaning, such as of surfaces, articles, etc. Consistent with various embodiments, the present disclosure may provide methods, products, kits, and systems that may be utilized to verify cleaning, and/or assist in verifying cleaning. Proper cleaning protocols, that are effectively implemented, can be a valuable tool in preventing, reducing, or controlling the presence, incidence, and spread of illness or harm that may be caused by infectious or harmful organisms (such as viruses, bacteria, spores, and the like) and/or other potentially harmful, or undesired, materials (e.g., hazardous drug trace contaminants, hazardous chemical contaminants, etc.). As such, it may be desirable for entities to be able to be confident that proper cleaning is occurring. However, surfaces may visually appear clean, but there is no way of telling whether a cleaning protocol is effective, if the cleaning protocol has actually been followed or carried out in an effective manner (for example, whether any cleaning that may have taken place has been thorough). In some implementations, the present disclosure may be utilized to evaluate the competency of the people performing the cleaning, and/or the effectiveness of the cleaning protocol itself.

Consistent with some implementations, the present disclosure may be utilized as a cleaning quality improvement tool, a cleaning efficacy verification tool, and/or a cleaning training tool. Accordingly, some embodiments consistent with the present disclosure may be utilized in connection with an organizations' quality improvement/risk management program with regard to the organization's cleaning protocol, staff effectiveness/thoroughness, and/or task confirmation. Further, in some implementations, embodiments consistent with the present disclosure may be utilized as a cleaning protocol/task training validation system, which may allow for measurement and return demonstration of training and task performance for individuals responsible for performing cleaning duties within an organization. According to some illustrative examples, embodiments consistent with the present disclosure may be utilized by organizations such as, but not limited to, healthcare entities (e.g., hospitals, clinics, physician offices, dental offices, nursing home/extended care facilities), commercial real estate (e.g., hotels, restaurants, office buildings and retail establishments, such as grocery stores, malls/stores), residential real estate (e.g., condominiums and apartment buildings), academic institutions (e.g., universities, colleges, high schools, elementary and pre-schools), government offices, as well as any and all spaces intended for public gatherings. It will be appreciated that implementations consistent with the present disclosure may find application in additional and/or alternative organizations and/or environments.

Consistent with some example embodiments, the present disclosure may generally allow cleaning efficacy to be determined by marking a surface and/or object prior to an anticipated cleaning, and then determining whether the surface and/or object has been sufficiently cleaned to remove the marking from the surface and/or object. Consistent with some implementations, marking the surface and/or object may be accomplished without leaving a readily and/or easily visibly observable indication. In some such implementations, the anticipated cleaning may not be specifically concentrated on removing a visually apparent marking from the surface and/or object.

According to various embodiments and implementations, concepts consistent with the present disclosure may be used for verifying whether a cleaning has taken place, verifying the efficacy of the cleaning, training workers relative to a cleaning protocol, validating a cleaning protocol, and/or selecting, designing, testing, etc., one or more possible cleaning protocols. For example, there can be a difference (perhaps a significant difference) between something appearing clean, and actually having a reduced presence of potentially harmful organisms and/or substances. Consistent with some implementations, the present disclosure may enable and/or provide a demonstration of a level of cleaning of a surface and/or object in which the cleaning was not specifically carried out to address a particular visibly apparent or noticeable soiling or “unclean” condition. It will be appreciated that embodiments consistent with the present disclosure may be utilized in other contexts and/or applications.

Continuing with the foregoing, according to an illustrative example embodiment, consistent with the present disclosure, a method for verifying cleaning may include applying a marking agent to a surface to be cleaned. The method may also include testing the surface for the presence of the marking agent on the surface after an expected cleaning of the surface. In general, the surface to be cleaned may be any surface and/or object. For example, the surface may include, but is not limited to, counters, tables, door handles, elevator buttons, equipment (e.g., lab equipment, IV drug administration equipment, food preparation equipment), bathroom fixtures, walls, floors, and/or any other surfaces for which it may be desirable to verify cleaning.

Consistent with some example implementations, it may be desirable to verifying the cleaning of the surfaces, overall. Such verification may be intended for evaluating an effectiveness of a cleaning protocol, whether the cleaning protocol was actually carried out, how well the cleaning protocol was adhered to and/or implemented, for training an individual in a cleaning protocol by providing demonstration of cleaning effectiveness, and/or for any other suitable use. As such, in some example embodiments it may be desirable that the marking agent may not be readily visually perceptible after application to the surface. As such, it may be less likely, for example, that any cleaning that takes place may not be concentrated around the presence of the marking agent and/or directed at removing the marking agent from the surface. Consistent with some such embodiments, when applied to the surface the marking agent may be generally colorless and odorless, and may not be readily visually apparent on the surface and/or may not be readily identified as a marking agent. Consistent with various embodiments, and as will be discussed in greater detail, the marking agent may be applied as a liquid and/or as a solid. As such, in some embodiments, the marking agent may be visually perceptible immediately after application to the surface, but may dry and/or otherwise become less readily visually perceptible on the surface after a period of time. Such implementations are considered consistent with the present disclosure.

As generally discussed, the marking agent may include a material that may be applied to a surface, such that the surface may subsequently be tested for the presence of the marking agent. As such, the marking agent may include a wide variety of materials. Consistent with some illustrative example embodiments, the marking agent may include an acidic material and/or a basic material. For example, in some particular example embodiments, the marking agent may include an acid, such as, but not limited to, citric acid, phosphoric acid, sulfuric acid, acetic acid, carbonic acid, adipic acid, peracidic acid, etc. Consistent with some illustrative example embodiments, the marking agent may generally be provided and/or formulated having a pH from about 2.0-2.7. It will be appreciated that other pH ranges may also suitably be implemented.

Further, in some example embodiments, the marking agent may include a component that may allow the surface to be tested for the presence of the marking agent, and may additionally include a carrier that may, for example, facilitate applying the marking agent to the surface, diminishing a characteristic of other components of the marking agent (e.g., decreasing an acidity of the marking agent, decreasing an odor associated with the marking agent), and/or increasing the overall volume of the marking agent (e.g., to reduce the cost, allow easier application, allow broader application, etc.). Consistent with various embodiments, any suitable carrier fluid may be utilized, including, but not limited to, water, methanol, ethanol, propanol (e.g., isopropanol), acetone, as well as any other suitable carrier fluids. In some illustrative example embodiments, the carrier fluid may include a liquid that may dry or evaporate relatively quickly. Upon such drying, the surface may retain at least a trace of the component of the marking agent that may be tested for (e.g., during the verification of cleaning). In a particular illustrative example embodiment, the marking agent may include a mixture of an acid and an alcohol.

In an example implementation, the marking agent may generally not damage, discolor, and/or otherwise alter the surface in an undesired and/or permanent manner. For example, when the marking agent includes an acid, the particular acid, the strength of the acid, and/or the degree of dilution in the carrier fluid may be selected such that the marking agent may not damage, discolor, and/or otherwise alter the surface in an undesirable manner. Similarly, the carrier fluid of the marking agent may be selected such that it may not damage, discolor, and/or alter the surface in an undesired manner. In some implementations, the marking agent may be selected and/or formulated based upon, at least in part, the nature of the surface to which the marking agent will be applied. For example, different marking agents may be selected and/or formulated for different intended surfaces (e.g., metal, wood, glass, stone, etc.). Accordingly, in some illustrative example embodiments, a variety of marking agents may be provided and utilized for different surfaces.

According to a particular illustrative example embodiment, the marking agent may include a mixture of isopropyl alcohol and phosphoric acid. In a particular embodiment, the marking agent may include a mixture of 99.9% isopropyl alcohol and 85% phosphoric acid prepare in a relative ration of 493.8 ml 99.9% isopropyl alcohol and 1.6 ml 85% phosphoric acid. The resultant marking agent may have a density of approximately 0.7889 g/cm³, and a pH in the range of about 2.0-2.7. According to another particular illustrative example, a marking agent may be prepared as a mixture of 498.4 ml of 99% methanol and 1.6 ml of 85% phosphoric acid. The resultant marking agent may have a density of about 0.7949 g/cm3, and a pH in the range of about 2.0-2.7. It will be appreciated that milliliter content is intended as an indication of the relative ratio make-up of the marking agent, and that the ultimate quantity of marking agent may vary.

Consistent with the present disclosure, any suitable method of applying the marking agent to the surface may be utilized. According to some implementations, applying the marking agent to the surface may include applying the marking agent generally uniformly to the surface, and/or applying the marking agent to a relatively large portion or extent of the surface. Consistent with such an implementations, cleaning of the entire surface treated with the marking agent may be verified. According to some implementations, applying the marking agent to the surface may include applying the marking agent to selected regions of the surface. Consistent with some such implementations, cleaning may be verified with respect to the specific selected regions of the surface to which the marking agent is applied. Such an approach may, for example, facilitate ensuring that particular portions of the surface (e.g., which may include critical portions of the surface and/or regions of the surface that may be often missed or skipped) have been cleaned, and/or may generally allow verification of cleaning while using less marking agent (e.g., as marking agent is only applied to specific regions of the surface).

In some illustrative example embodiments, applying the marking agent to the surface may include spraying the marking agent on to the surface. For example, in some embodiments, the marking agent may be applied using a spray bottle, or similar dispenser, which may apply a mist or droplets of the marking agent to the surface. As such, the marking agent may be dispersed over a portion of the surface. In a particular embodiment, a spray bottle may be selected for providing a 0.08 ml spray of marking agent to the surface for each actuation of the spray mechanism. Such a metered application of the marking agent may allow a sufficient quantity of marking agent to be applied to the surface for testing, while reducing the use of unnecessary quantities of the marking agent (e.g., which may generally be wasteful and/or potentially damaging and/or altering the surface in an undesired manner).

In some illustrative example embodiments, applying the marking agent to the surface may include wiping the surface with an applicator including the marking agent. Any suitable applicator for wiping the marking agent onto the surface may be utilized. Example, of suitable applicators may include woven or non-woven textile products, paper towels, sponges, absorbent applicator pads, swabs, as well as any other suitable product for applying the marking agent to the surface. In some example embodiments, wiping the surface with an applicator may include dispensing the marking agent onto the applicator and wiping the surface with the applicator. For example, the marking agent may be dispensed onto the applicator by spraying the applicator with a spray bottle containing the marking agent, by pouring the marking agent onto the applicator from a container (such as a bottle, or the like), and/or by any other suitable mechanism for dispensing the marking agent onto the applicator. In some illustrative example embodiments, a spray bottle may be configured to provide a metered dosage of marking agent, e.g., which may be appropriate for applying the marking agent to a surface using an applicator. Consistent with such implementations, the applicator may include any suitable purpose specific applicator and/or ad hoc applicator (e.g., such as a rag, paper towel, etc.). In some example implementations, wiping the surface with an applicator may include wiping the surface with a pretreated applicator including the marking agent. For example, the marking agent may be provided as pre-packaged applicators that have already been moistened with the marking agent. As such, the applicator may simply be removed from a packaging, and then wiped across a surface to be treated with the marking agent. Examples of such pre-packaged, pre-moistened applicators may include, but are not limited to, individually and/or bulk packaged swabs, individually or bulk packaged (e.g., in a dispensing canister) towels/towelettes, or the like. Consistent with one illustrative example embodiment, a pre-packaged and/or pre-moistened applicator may be preloaded (e.g., pre-moistened) with approximately 300 mg of the marking agent. It will be appreciated that an applicator may be preloaded with a greater or lesser amount of the marking agent (e.g., depending upon the size of the applicator, etc.).

In some implementations, applying the marking agent to the surface may include dusting the surface with the marking agent. For example, rather than be provided in liquid form, the marking agent may include a powder or particulate material. Consistent with such an implementation, the marking agent itself may be powder or particulate material and/or the marking agent may include a component that can be tested for (as described in greater detail below), which may be absorbed into and/or coated onto a powder or particulate carrier material. Consistent with some such embodiments, the powder marking agent may be dusted onto the surface using a dusting brush, a shaker applicator, and/or using other similarly suitable means.

Consistent with an illustrative example embodiment, testing the surface for the presence of the marking agent may include testing the surface after an anticipated cleaning of the surface. For example, when verifying that cleaning has occurred and/or the effectiveness or thoroughness of cleaning, the marking agent can be applied to the surface prior to an anticipated cleaning. Such an anticipated cleaning may be based upon a cleaning schedule or expectation (for example, the surface and/or area including the surface may be scheduled and/or expected to be cleaned daily, weekly, etc.), and/or may be based on another suitable basis. Similarly, when evaluating a cleaning protocol, or providing training or demonstration regarding a cleaning protocol, the marking agent may be applied to the surface before the training protocol is actually implemented. Depending upon the nature of the marking agent, the marking agent may be applied to the surface prior to the anticipated cleaning by a sufficient amount of time to allow the marking agent to dry/evaporate the carrier fluid, as necessary. Additionally, in some embodiments the marking agent may have a useful lifespan, after which testing for the marking agent may not provide reliable results (e.g., even if the surface has not been cleaned, after the useful lifespan of the marking agent, testing the surface may not positively indicate the presence of the marking agent). The useful lifespan of the marking agent may be a characteristic of the marking agent itself, or a time period over which people or object contacting the surface may be expected to remove or disrupt the marking agent, compromising the ability to reliable detect the presence of the marking agent, regardless of any cleaning that may, or may not, have occurred. As such, the anticipated cleaning of the surface may be within the useful lifespan of the marking agent. Consistent with the present disclosure, the useful lifespan of the marking agent may include several hours, several days, several weeks, or even longer.

Consistent with the present disclosure, testing the surface for the presence of the marking agent may include contacting the surface with an indicator agent. In some example embodiments, the indicator agent may provide a visually perceptible indication of the presences of the marking agent on the surface. For example, if marking agent is present on the surface (e.g., due to a lack of cleaning, ineffective cleaning, and/or an ineffective cleaning protocol), when the surface is contacted with the indicator agent, the indicator agent may provide a visually perceptible indication of the presence of the marking agent. As such, additional equipment, testing, and/or tools may not be necessary to verify the cleaning/thoroughness of the cleaning. As noted above, in some implementations, the marking agent may only be applied to selected regions and/or portions of the surface. In such implementations, contacting the surface with the indicator agent may only require contacting the selected regions and/or portions of the surface with the indicator agent. Consistent with the foregoing example embodiments, detecting the presence of the marking agent on the surface after an expected cleaning of the surface may indicate at least one of a lack of the expected cleaning and an ineffectiveness of the expected cleaning.

As generally discussed above, in some illustrative example embodiments the marking agent may include an acid. Consistent with some such embodiments, the indicator agent may include an acid/base indicator. As is known, some acid/base indicators may provide a visual indication based upon, at least in part, a pH of a material reacting with the acid/base indicator (e.g., a pH of the marking agent). For example, the acid/base indicator may change color (which may include presenting a color from an initially colorless state and/or becoming colorless from an initially colored state) based upon a pH of a material reacting with the acid/base indicator being above a threshold, below a threshold, within a pH range, or the like. Some acid/base indicators and/or acid/base indicator systems (e.g., which may include a plurality of acid/base indicators together and/or used in conjunction with one another) may present a color indicative of the pH of the material reacting with the acid/base indicator or acid/base indicator system. Some illustrative example acid/base indicators may include, but are not limited to, bromophenol blue, bromothymol blue, bromocresol green, thymolphthalein, methyl orange, and the like.

Consistent with some illustrative example embodiments, the indicator agent may include an acid/base indicator in a liquid form. In some illustrative example embodiments, the indicator agent may include an acid/base indicator in combination with a liquid carrier. In some implementations, the liquid carrier may, at least in part, dissolve marking agent present on the surface to facilitate a reaction between the marking agent and an acid/base indicator of the indicator agent. In some implementations, the liquid carrier may perform additional functions, such as ensuring that the indicator agent remains outside of a detection pH range or level prior to reacting with the marking agent.

Consistent with one illustrative example embodiment, the indicator agent may include a solution of 344 ml of 99.9% isopropyl alcohol, 332 ml of distilled water, 136 ml of 0.04% solution of bromothymol blue (which may, in some embodiments be formed from 0.05 g bromothymol blue powder, 8.70 ml of 0.01N sodium hydroxide solution, and 127.30 ml distilled water), 136 ml of 0.04% solution of bromophenol blue (which may, in some embodiments be formed from 0.05 g bromophenol blue powder, 8.11 ml of 0.01N sodium hydroxide solution, and 127.89 ml of distilled water), and 52 ml of 0.01N sodium hydroxide solution (which may, in some embodiments be formed from 0.02 g of 99.9% sodium hydroxide and 52 ml of distilled water). The resulting indicator agent may have a density of about 0.926 g/cm³, and a pH of about 8.0-8.5. Consistent with an illustrative example embodiment, the indicator agent may be capable of detecting the presence of a marking agent having a pH within the range of about 1.9-6.0. It will be appreciated that milliliter content is intended as an indication of the relative ratio make-up of the marking agent, and that the ultimate quantity of marking agent may vary. Additionally, it will be appreciated that other indicator agent formulations may be utilized, including other acid/base indicators, other solvents, and the like. For example, other solvents, such as methanol, ethanol, etc., may be utilized.

In a similar manner as discussed with respect to the marking agent, in general the indicator agent may be formulated and/or selected such that contacting the surface with the indicator agent is unlikely to damage, discolor, or alter the surface in an undesired manner. As such, the components of the indicator agent (e.g., the acid/base indicator, the carrier, etc.) may be selected based upon the nature of the surface (e.g., metal, glass, wood, stone, etc.). Further, in some implementations, a variety of indicator agents may be provided. In such an implementation, an appropriate indicator agent of the variety of indicator agents may be selected based upon, at least in part, the nature of the surface. The surface may be contacted with selected indicator agent for testing for the presence of the marking agent.

As generally discussed above, in some embodiments the marking agent may be applied generally uniformly across the surface and/or over a relatively large extent of the surface. Consistent with such an implementation, contacting the surface with the indicator agent may include contacting any portion of the surface to which the marking agent may be likely to have been applied with the indicator agent. This may include contacting a substantial portion of the surface with the indicator agent. However, it will be appreciated that contacting a substantial portion of the surface with the indicator agent may leave residual indicator agent on the surface. It may be desirable to remove the indicator agent from the surface, e.g., to prevent contamination of people or items coming in contact with the surface. As such, it may be desirable to clean any portions of the surface that have been contacted with the indicator agent. This may not be of concern if, for example, the cleaning verification is utilized in the context of determining an effectiveness of a cleaning protocol or as part of a cleaning training program. However, if the cleaning verification is utilized in the context of determining if a surface has been properly cleaned (or cleaned at all), it may be undesirable to create a situation in which a surface that has been determined to have been properly cleaned must be re-cleaned. As such, it may be desirable to only contact select areas of the surface with the indicator agent, which may be re-cleaned relatively easily. Further, as discussed above, in some implementations that marking agent may be applied to select regions or portions of the surface. In such an implementation, the same select regions or portions of the surface may be contacted with the indicator agent.

According to an illustrative example embodiment, contacting the surface with an indicator agent may include spraying the surface with the indicator agent. For example, in some embodiments, the indicator agent may be applied to the surface using a spray bottle, or similar dispenser, which may apply a mist or droplets of the indicator agent to the surface. As such, the indicator agent may be dispersed over a portion of the surface. The indicator agent applied to the surface may react with any marking agent on the surface, resulting in a visibly perceptible indication of the presence of the marking agent (e.g., in the form of a color change of the indicator agent on the surface) In a particular embodiment, a spray bottle may be selected for providing a 0.08 ml spray of the indicator agent to the surface for each actuation of the spray mechanism. Such a metered application of the indicator agent may allow the surface to be contacted with a sufficient quantity of indicator agent for testing for the presence of the marking agent, while reducing the use of unnecessary quantities of the indicator agent (e.g., which may generally be wasteful and/or potentially damaging and/or altering the surface in an undesired manner).

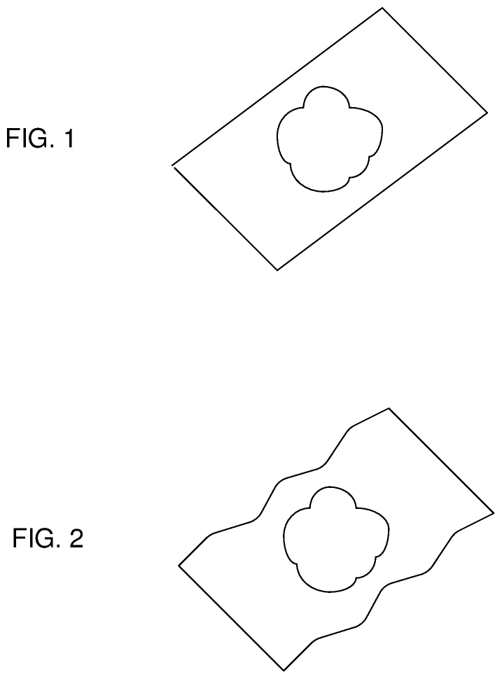

In some embodiments, contacting the surface with an indicator agent may include wiping the surface with an applicator including the indicator agent. When the applicator including the indicator agent is wiped across a portion of the surface including marking agent, the indicator agent on the applicator may react with the marking agent on the surface resulting in a visually perceptible indication of the presence of the marking agent. In some example embodiments, the visually perceptible indication may include, for example, a quantity of indicator agent being transferred to the surface where the quantity of indicator agent may react with the marking agent on the surface. As diagrammatically shown in FIG. 1, this may result in color change (or the presence of color-changed indicator agent) being present on the surface (e.g., in the region corresponding to the marking agent). In some embodiments, wiping a portion of the surface including the marking agent may result in a quantity of the marking agent being transferred from the surface to the applicator. As diagrammatically shown in FIG. 2, the quantity of marking agent transferred to the applicator may react with the indicator agent on the applicator, resulting in a color change of at least a portion of the applicator corresponding to the reacted indicator agent on the applicator.

In some illustrative example embodiments consistent with the present disclosure, wiping the surface with an applicator may include dispensing the indicator agent onto the applicator and wiping the surface with the applicator. Any suitable applicator may be utilized for wiping the surface with the indicator agent. Example, of suitable applicators may include woven or non-woven textile products, paper towels, sponges, absorbent applicator pads, swabs, as well as any other suitable product for with the surface with the indicator agent. In some example embodiments, wiping the surface with an applicator may include dispensing the indicator agent onto the applicator and wiping the surface with the applicator. For example, the indicator agent may be dispensed onto the applicator by spraying the applicator with a spray bottle containing the indicator agent, by pouring the indicator agent onto the applicator from a container (such as a bottle, or the like), and/or by any other suitable mechanism for dispensing the indicator agent onto the applicator. In some particular illustrative example embodiments, the indicator agent may be sprayed from a spray bottle providing a specific metered dosage of the indicator, e.g., as may be appropriate for use with an applicator. Consistent with such implementations, the applicator may include any suitable purpose specific applicator and/or ad hoc applicator (e.g., such as a rag, paper towel, etc.).

In some example implementations, wiping the surface with an applicator may include wiping the surface with a pretreated applicator including the indicator agent. For example, the indicator agent may be provided as pre-packaged applicators that have already been moistened with the indicator agent. As such, the applicator may simply be removed from a packaging, and then wiped across a surface to contact the surface with the indicator agent, thereby testing for the presence of the marking agent. Examples of such pre-packaged, pre-moistened applicators may include, but are not limited to, individually and/or bulk packaged swabs, individually or bulk packaged (e.g., in a dispensing canister) towels/towelettes, or the like. Consistent with a particular illustrative example embodiment, an applicator may be pre-packaged and/or pretreated applicator may be preloaded with 500 mg of the indicator agent. It will be appreciated that an applicator may be preloaded with a greater or lesser amount of indicator agent, e.g., depending on a size and/or type of applicator.

According to some illustrative example embodiments, the present disclosure may provide a cleaning protocol verification kit. Generally, the kit may include a marking agent dispenser configured for applying a marking agent to a surface. The cleaning protocol verification kit may also generally include an indicator agent dispenser configured for contacting an indicator agent with the surface. The indicator agent may provide a visually perceptible indication of the presences of the marking agent on the surface for one or more of training users to implement a cleaning protocol and verifying an efficacy of cleaning of the surface.

As generally discussed above, in an illustrative example embodiment, the marking agent may include an acid, and the indicator agent may include an acid base indicator. Furthermore, the acid/base indicator may be configured to provide a visually perceptible change when contacted with the marking agent. As such, in some embodiments the kit may allow cleaning verification without the need for additional equipment. Further, in some implementations, the kit may include multiple marking agent dispensers including multiple different marking agents. The multiple marking agents may include different acids and/or may include different carrier fluids. The different acids and/or different carrier fluids may be formulated to provide suitable use on different surfaces (e.g., metal, glass, plastic, wood, etc.), without causing undesired damage, discoloration, and/or alteration of the surface. In a generally corresponding manner, a kit consistent with an example embodiment of the present disclosure may include multiple indicator agent dispensers including multiple different indicator agents. The multiple indicator agents may include different acid/base indicators and/or may include different carrier fluids. The different acid/base indicators and/or different carrier fluid may be formulated to provide suitable use on different surfaces (e.g., metal, glass, plastic, wood, etc.), without causing undesired damage, discoloration, and/or alteration of the surface.

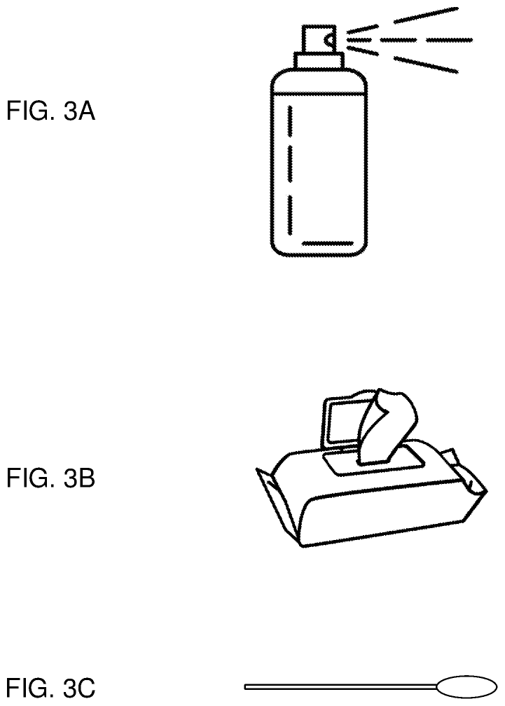

Consistent with an example embodiment, and as diagrammatically shown in FIG. 3A, the marking agent dispenser may include a spray bottle containing the marking agent In a particular embodiment, the spray bottle containing the marking agent may be configured to dispense a metered amount of marking agent onto the surface. For example, the spray bottle may be configured to dispense a 0.08 ml dose of the marking agent for each actuation of the spray mechanism. Further, in some implementations, the marking agent dispenser may include a spray bottle containing the marking agent, in which the spray bottle may be configured to dispense a metered amount of marking agent onto an applicator, such as a wipe/cloth/towel/etc., or swab, as diagrammatically shown in FIGS. 3B-3C. In some such implementation, the metered dose may be greater than, for example, a metered dose intended to be applied directly to the surface (e.g., to account for any quantity of the marking agent that may be absorbed by the applicator and/or not transferred from the applicator to the surface). In some such implementations, the kit may additionally include “blank” applicators (e.g., applicators not having the marking agent pre-applied). The marking agent may be applied to the blank applicators, and subsequently wiped across the surface to apply the marking agent to the surface. As diagrammatically shown in FIGS. 3B-3C, examples of blank applicators may include a woven and/or non-woven fabric (e.g., which may in some implementations be used a multi-use applicator for applying the marking agent to multiple surfaces and/or over the course of multiple use occasions), wipes, sponges, towelettes, swabs, and the like. In other implementations, the spray bottle may be utilized to apply the marking agent to any suitable applicator (rag, paper towel, wipe, etc.) that may be provided by the user. Further, in some implementations, the marking agent dispenser may include an applicator pretreated with the marking agent. As generally discussed above, applicators pretreated with the marking agent may be individually packaged and/or bulk packaged (e.g., multiple applicators in a common package, which may, in some cases, be at least partially or completely re-sealable). Pretreated applicators may take any suitable form, including, but not limited to, wipes (e.g., paper-based, woven or non-woven fabric, natural or synthetic materials), sponges, swabs (e.g., cotton/synthetic cotton on a stick), or the like.

Consistent with an example embodiment, the indicator agent dispenser may include a spray bottle containing the indicator agent In a particular embodiment, the spray bottle containing the indicator agent may be configured to dispense a metered amount of indicator agent onto the surface. For example, the spray bottle may be configured to dispense a 0.08 ml dose of the indicator agent for each actuation of the spray mechanism. Further, in some implementations, the indicator agent dispenser may include a spray bottle containing the indicator agent, in which the spray bottle may be configured to dispense a metered amount of indicator agent onto an applicator. In some such implementation, the metered dose may be greater than, for example, a metered dose intended to be applied directly to the surface (e.g., to account for any quantity of the indicator agent that may be absorbed by the applicator and/or not transferred from the applicator to the surface). In some such implementations, the kit may additionally include “blank” applicators (e.g., applicators not having the indicator agent pre-applied). The indicator agent may be applied to the blank applicators, and subsequently wiped across the surface to contact the indicator agent with any marking agent that may be present on the surface. As diagrammatically shown in FIGS. 3B-3C, examples of blank applicators may include, but are not limited to, a woven and/or non-woven fabric (e.g., which may in some implementations be used a multi-use applicator for applying the marking agent to multiple surfaces and/or over the course of multiple use occasions), wipes, sponges, towelettes, swabs, and the like. In other implementations, the spray bottle may be utilized to apply the indicator agent to any suitable applicator (rag, paper towel, wipe, etc.) that may be provided by the user. Further, in some implementations, the indicator agent dispenser may include an applicator pretreated with the indicator agent. As generally discussed above, applicators pretreated with the indicator agent may be individually packaged and/or bulk packaged (e.g., multiple applicators in a common package, which may, in some cases, be at least partially or completely re-sealable). Pretreated applicators may take any suitable form, including, but not limited to, wipes (e.g., paper-based, woven or non-woven fabric, natural or synthetic materials), sponges, swabs (e.g., cotton/synthetic cotton on a stick), or the like.

A variety of illustrative example embodiments have been described, each including a variety of features, concepts, and arrangements. It will be appreciated that features, concepts, and arrangements disclosed in the context of one, or several, discrete embodiments are susceptible to application in other embodiments, and/or susceptible to combination with features, concepts, and/or arrangements discussed relative to multiple different embodiments. Herein, such combination of features, concepts, and arrangements from the several embodiments is expressly intended to be within the scope of the present disclosure. Similarly, a variety of feature, advantages, implementations, and embodiments have been described herein. However, it will be appreciated that the foregoing description and the depicted embodiments are only intended for the purpose of illustration and explanation, and should not be construed as a limitation on the present invention. It will be appreciated that the features and concepts associated with the various embodiments are susceptible to combination with features and concepts of other disclosed embodiments. Additionally, it will be appreciated that the concepts embodied by the description and illustration are susceptible to variation and modification, all of which are intended to be encompassed by the present invention.

What is claimed is:

1. A method comprising:
applying a marking agent to a surface to be cleaned;
testing the surface for the presence of the marking agent on the surface after an expected cleaning of the surface.

2. The method according to claim 1, wherein applying the marking agent to the surface includes one or more of spraying the marking agent on to the surface, wiping the surface with an applicator including the marking agent, and dusting the surface with the marking agent.

3. The method according to claim 2, wherein wiping the surface with an applicator includes one or more of dispensing the marking agent onto the applicator and wiping the surface with the applicator and wiping the surface with a pretreated applicator including the marking agent.

4. The method according to claim 1, wherein the marking agent is not readily visually perceptible after application to the surface.

5. The method according to claim 1, wherein the marking agent includes an acid.

6. The method according to claim 5, wherein the marking agent includes a mixture of the acid and a carrier fluid.

7. The method according to claim 1, wherein testing includes testing after an anticipated cleaning of the surface.

8. The method according to claim 1, wherein testing the surface for the presence of the marking agent includes contacting the surface with an indicator agent.

9. The method according to claim 8, wherein contacting the surface with an indicator agent includes one or more of spraying the surface with the indicator agent and wiping the surface with an applicator including the indicator agent.

10. The method according to claim 9, wherein wiping the surface with an applicator includes one or more of dispensing the indicator agent onto the applicator and wiping the surface with the applicator and wiping the surface with a pretreated applicator including the indicator agent.

11. The method according to claim 8, wherein the indicator agent provides a visually perceptible indication of the presences of the marking agent on the surface.

12. The method according to claim 8, wherein the marking agent includes an acid, and the indicator agent includes an acid/base indicator.

13. The method according to claim 1, wherein detecting the presence of the marking agent on the surface after an expected cleaning of the surface indicates at least one of a lack of the expected cleaning and an ineffectiveness of the expected cleaning.

14. A cleaning protocol verification kit comprising:
a marking agent dispenser configured for applying a marking agent to a surface; and
an indicator agent dispenser configured for contacting an indicator agent with the surface, the indicator agent providing a visually perceptible indication of the presences of the marking agent on the surface for one or more of training users to implement a cleaning protocol and verifying an efficacy of cleaning of the surface.

15. The cleaning protocol verification kit according to claim 14, wherein the marking agent dispenser includes a spray bottle containing the marking agent and configured to dispense a metered amount of marking agent one of onto the surface and onto an applicator.

16. The cleaning protocol verification kit according to claim 14, wherein the marking agent dispenser includes an applicator pretreated with the marking agent.

17. The cleaning protocol verification kit according to claim 14, wherein the indicator agent dispenser includes a spray bottle containing the indicator agent and configured to dispense a metered amount of the indicator agent one of onto the surface and onto an applicator.

18. The cleaning protocol verification kit according to claim 14, wherein the indicator agent dispenser includes an applicator pretreated with the indicator agent.

19. The cleaning protocol verification kit according to claim 14, wherein the marking agent includes an acid, and the indicator agent include an acid/base indicator.

20. The cleaning protocol verification kit according to claim 19, wherein the acid/base indicator is configured to provide a visually perceptible change when contacted with the marking agent.
